(19) United States
(12) Patent Application Publication
(10) Pub. No.: US 2008/0194491 A1
(43) Pub. Date: Aug. 14, 2008

(54) Methods of Diagnosing, Preventing, and Treating Early Onset of Pulmonary Hypertension

(76) Inventors: Duncan J. Stewart, Toronto (CA); Saeid Babaei, Toronto (CA); David Courtman, Toronto (CA)

Correspondence Address: THE WEBB LAW FIRM, P.C., 700 KOPPERS BUILDING, 436 SEVENTH AVENUE, PITTSBURGH, PA, 15219, US

(21) Appl. No.: 10/540,718

(22) PCT Filed: Dec. 24, 2003
(86) PCT No.: PCT/CA2003/002007

§ 371 Date: Dec. 21, 2006

Related U.S. Application Data
(60) Provisional application No. 60/435,861, filed on Dec. 24, 2002.

(51) Int. Cl. A61K 38/22; A61K 31/195; A61P 11/00; A61K 38/06

(52) U.S. Cl. 514/18; 514/561; 514/12

(57) ABSTRACT

Early onset of pulmonary disorders such as pulmonary hypertension are treated or prevented by administration of an apoptosis inhibitor or a survival factor. Early onset of such disorders may be diagnosed by assessing apoptosis in lung tissue.

### CROSS-REFERENCE TO RELATED APPLICATION

This application claims priority from U.S. Patent Application Ser. No. 60/435,861 filed Dec. 24, 2002 and currently pending. The entire disclosure of that application is incorporated herein by reference.

### FIELD OF THE INVENTION

This invention relates to medical treatments and diagnosis and compositions and procedures useful diagnosing, preventing and treating pulmonary hypertension.

### BACKGROUND OF THE INVENTION

Pulmonary hypertension is associated with significant morbidity and mortality, yet therapeutic options remain limited. Previous attempts to treat pulmonary hypertension have involved the use of prostacyclin, using continuous administration, but this is a difficult and expensive procedure, liable to give rise to side effects.

Dysfunctional endothelial cells have a central and critical role in the initiation and progression of severe pulmonary hypertension. The elucidation of the mechanisms involved in the control of endothelial cell proliferation and cell death in the pulmonary vasculature, therefore, is fundamentally important in the pathogenesis of pulmonary hypertension. Recent research has determined that primary pulmonary hypertension is a disease caused by somatic mutations in the bone morphogenic protein receptor 2 (BMPR2), a member of the TGFβ superfamily. It is believed that the loss of cell growth control mechanisms allows for the clonal expansion of endothelial cells from a single cell that has acquired a selective growth advantage (Tudor, R M et al., Clin Chest Med 22(3):405-18, 2001). It is generally accepted that, late in its course, severe pulmonary hypertension is associated with the occlusion or loss of a substantial portion of the pulmonary microvessels and that this is a major contributor to the increased pulmonary vascular resistance that characterizes this disease. It is hypothesized that this is due to the body responding to acute pulmonary hypertension with defense mechanisms including apoptosis (Gotoh, N, J Appl Physiol 88(2):518-26, 2000). It is believed that inducing apoptosis can cause the reversal of pathology (Rabinovitch, M, Clin Chest Med 22(3):43349, 2001). For example, it is believed that elastase inhibitors, which reduce extracellular matrix remodeling and β₃ integrin signaling, lead to increased smooth muscle cell apoptosis, and that this may contribute to reversal of advanced pulmonary vascular disease (Cowan, K N, Nat Med 6(6):698-702, 2000).

However, the present inventors have now found a new approach to detecting, treating, and preventing pulmonary hypertension that runs contrary to these hypotheses.

### SUMMARY OF THE INVENTION

The present invention is based upon the finding that loss of pulmonary microvessels is an early event in pulmonary hypertension. The present inventors have found that apoptosis is both a major mechanism in the early pathogenesis of pulmonary hypertension and a new target for therapy. Specifically, the present inventors have found 1) that, contrary to the accepted doctrine, endothelial cell (EC) apoptosis is an early event in the onset of pulmonary hypertension, preceding the increase in pulmonary pressures; 2) that selective pulmonary EC apoptosis is associated with the early loss of pulmonary microvessels and 3) that inhibition of apoptosis prevents pulmonary hypertension by preserving EC integrity at select levels of the vasculature reducing the loss of pulmonary microvessels.

Thus the present invention teaches a process for alleviating the symptoms of pulmonary disorders in a mammal, comprising administration of an effective amount of a factor selected from the group consisting of an apoptosis inhibitor and a survival factor to the pulmonarysystem of a mammal to alleviate a pulmonary disorder or symptoms thereof.

In another embodiment, the invention teaches a method for preventing symptoms of pulmonary disorders in a mammal, comprising administration of an apoptosis inhibitor to the pulmonary system of a mammal to prevent a pulmonary disorder or symptoms thereof.

The pulmonary disorder may be pulmonary hypertension. The mammal may be human. The apoptosis inhibitor may be administered early in the onset of the disorder.

In an embodiment, the apoptosis inhibitor may be delivered using viable, transfected mammalian cells, said transfected mammalian cells containing at least one expressible trans-gene coding for an apoptosis inhibitor or survival factor. The mammalian cells may be selected from the group consisting of dermal fibroblasts, smooth muscle cells, progenitor cells, stem cells, or endothelial cells.

The invention also teaches apoptosis inhibitors useful for administration to a mammalian patient's pulmonary system to alleviate the symptoms of a pulmonary disorder in said patient.

In an embodiment, the apoptosis inhibitor may be selected from the group consisting of Z-Asp, Z-VAD, VEGF, Bcl-2, Bcl-xL, acetyl-DEVD-aldehyde inhibitor, acetyl-YVAD-aldehyde, acetyl-YVAD-chloromethylketone, Boc-D-(benzyl) chloromethylketone, crmA, Zn²⁺, aurintricarboxylic acid, cytochalasin B, NO, eNOS (endothelial nitric oxide synthase), iNOS (inducible nitric oxide synthase), nNOS (neuronal nitric oxide synthase), NO-donor compounds, Ang1 (Angiopoietin-1), Akt, AIP (apoptosis inhibitor protein) and BMP (bone morphogenetic protein).

The invention also teaches processes for early diagnosis of a pulmonary disorder in a mammal, comprising assessing apoptosis in the pulmonary system of a mammal, wherein apoptosis is indicative of early onset of said pulmonary disorder. The pulmonary disorder may be pulmonary hypertension. The diagnosis may be carried out using a caspase activity or immunoreactivity assessment.

The invention is further described for illustrative purposes, in the following specific, non-limiting Examples.

### BRIEF DESCRIPTION OF THE DRAWINGS

The invention will now be described, by way of example only, with reference to the accompanying drawings, in which

FIG. 1 is a pictograph illustrating apoptosis by TUNEL fluorescent assay using confocal microscopy, counterstained with DAPI to show nuclei, 3 days, and two or three weeks after the administration of monocrotaline (MCT).

FIG. 2 is a bar graph showing the percentage of TUNEL positive cells for untreated and MCT treated cells at 3, 14, and 21 days post treatment.

FIG. 3 is a bar graph showing caspase-3 activity in whole lung lysates at 3, 7, 14, and 21 days following MCT treatment.

FIG. 4 is a pictograph showing representative examples of active caspase-3 immunostaining of lung sections harvested 2 weeks after MCT alone (A) or together with cell-based gene transfer of VEGF (B).

FIG. 5 is a bar graph showing apoptosis of vascular cells at 1, 2, 3 and 4 weeks after MCT treatment alone or MCT plus Ang1.

FIG. 6 is a pictograph which shows imaging of the pulmonary arteriole bed for normal (6A), MCT treated (6B), and MCT with eNOS treated rats (FIG. 6C).

FIG. 7 is a graph showing RVSP at 14 and 28 days after MCT injection (A) and (B) medial area of pulmonary arterial vessels <30 μm and 30 to 60 μm in external diameter at 28 days for lungs from animals treated with MCT and pcDNA-transfected or pVEGF-transfected smooth muscle cells.

FIG. 8 is a bar graph illustrating the effect of Z-Asp on RSVP in MCT treated rats at 14 and 21 days.

FIG. 9 is a graph showing the effect of cell-based gene transfer of Ang1 on survival (FIG. 9A) and right ventricular systolic pressure (FIG. 9B, RVSP) in the rat MCT model of pulmonary hypertension.

FIG. 10 is a time line showing the administration of Z-Asp or Z-VAD in relation to MCT and time of sacrifice.

FIG. 11 is a graph showing the effect of Z-Asp or Z-VAD on the progression of apoptosis, 24, 48 and 72 hours post last dose of inhibitor based on the TUNEL assay.

FIG. 12A shows the elevation in right ventricular systolic pressure (RSVP) 21 days post MCT administration.

FIG. 12B shows the effect of Z-Asp on RSVP in MCT treated rats.

FIG. 13 is a bar graph illustrating the effect of BMP on apoptosis in human pulmonary artery epithelial cells treated with TNF.

FIG. 14 is a bar graph illustrating the effect of Z-Asp on RV/LV ratio.

FIG. 15 is a graph illustrating the lack of effect of BMP on cell proliferation.

FIG. 16 is a photomicrograph showing fluorescence microangiography of the pulmonary microcirculation 21 days following treatment with MCT or MCT+Z-Asp.

FIG. 17 is a pictograph illustrating apoptosis in cells treated with 15% FCS and in serum deprived cells as determined by flow cytometry.

FIG. 18 is a pictograph illustrating apoptosis in untreated cells, BMP2 treated cells and BMP7 treated cells as determined by flow cytometry.

FIG. 19 is a bar graph illustrating the effect of BMP on apoptosis induced by serum withdrawal.

FIG. 20A is pictograph which shows imaging of cells treated with TNF.

FIG. 20B is a pictograph which shows image of cells treated with TNF and BMP.

FIG. 20C is a graph illustrating the effect of BMP on apoptosis induced by TNF.

### DESCRIPTION OF EMBODIMENTS

The present inventors have found that programmed endothelial cell death (apoptosis) presents a novel target in the prevention and treatment of pulmonary hypertension, including during the early onset of pulmonary hypertension.

One aspect of the present invention is the treatment of pulmonary hypertension (PH) using apoptosis inhibitors. Primary pulmonary hypertension (PPH) and other causes of pulmonary arterial hypertension are associated with severe abnormalities inendothelial function, which play a critical role in its pathogenesis.

The present invention provides, from a second aspect, a method of alleviating the symptoms of PPH (and other causes of PH) which comprises administering to the pulmonary system of a patient suffering therefrom transfected cells (e.g. mammalian fibroblast cells from dermal or other origins, endothelial cells or progenitor cells derived from bone marrow or isolated from the systemic circulation), said transfected cells including at least one expressible trans-gene coding for an apoptosis inhibitor for release thereof into the pulmonary circulation.

The present invention provides, in another aspect, a method of alleviating the symptoms of PPH (and other causes of PH) which comprises administering to the pulmonary system of a patient suffering therefrom liposomes, viral vectors, or other gene transfer vectors including at least one expressible trans-gene coding for an apoptosis inhibitor for release thereof into the pulmonary circulation.

The present invention provides, in another aspect, a method of alleviating the symptoms of PPH (and other causes of PH) which comprises administering to the pulmonary system of a patient suffering therefrom, by any of the methods contemplated herein, of endothelial cell survival factors (i.e. Ang1, VEGF) which can inhibit apoptosis in endothelial cells but not in smooth muscle cells. This method allows for reversal of medial hypertrophy.

### Inhibiting Apoptosis

A wide variety of anti-apoptosis factors, including trans-genes encoding anti-apoptosis factors can be used in the processes and products of the present invention. Thus, anti-apoptosis factors can be administered directly or expressed by trans-genes and delivered by the circulation of other body organs downstream of the lungs. Transfected cells lodged in the lung and containing trans-genes expressing such factors and other products will act as a paracrine source of the appropriate factor. As used herein, “apoptosis inhibitors” and “anti-apoptosis factors” and the like refer generally to factors which decrease cell death or increase cell survival or proliferation.

DNA sequences constituting the genes for these apoptosis inhibitors are known, and they can be prepared by the standard methods of recombinant DNA technologies (for example enzymatic cleavage and recombination of DNA), and introduced into mammalian cells, in expressible form, by standard genetic engineering techniques such as those known in the art (e.g. viral transfection, electroporation, lipofection, use of polycationic proteins, etc). VEGF is one preferred apoptosis inhibitor, on account of the greater experience with this factor and its level of effective expression in practice. ANG-1 is another preferred apoptosis inhibitor.

Protease gene families (Martin, S. J. et al., Cell (1995) 82:349-352), intracellular second messengers (Kroemer, G. et at., FASEB J (1995) 9:1277-1287), tumor suppressor genes (Hafifer, R. et al., Curr Op Gen Dev (1995) 5:84-90), and negative regulatory proteins are known that counteract apoptotic cell death (Hockenbery, D. et al., Nature (1990) 348:334-336).

Apoptosis inhibitors for use in the invention include several members of a gene family of inhibitors of apoptosis related to the baculovirus IAP gene (Birnbaum, M. J. et al., J Virology (1994) 68:2521-2528; Clem, R. J. et al., Mol Cell Biol (1994) 14:5212-5222) which have been identified in Drosophila and mammalian cells (Duckett, C. S. et al., EMBO J (1996) 15:2685-2694; Hay, B. A. et al., Cell (1995) 83:1253-1262; Liston, P. et al., Nature (1996) 379:349-353; Rothe, M. et al., Cell (1995) 83:1243-1252; Roy, N. et al., Cell (1995) 80:167-178). These molecules are highly conserved evolutionarily; they share a similar architecture organized in two or three approximately 70 amino acid amino terminus Cys/His baculovirus IAP repeats (BIR) and by a carboxy terminus zinc-binding domain, designated RING finger (Duckett, C. S. et al., EMBO J (1996) 15:2685-2694; Hay, B. A et al., Cell (1995) 83:1253-1262; Liston, P. et al., Nature (1996) 379:349-353; Rothe, M. et al., Cell (1995) 83:1243-1252; Roy, N. et al., Cell (1995) 80:167-178). Recombinant expression of IAP proteins blocks apoptosis induced by various stimuli in vitro (Duckett, C. S. et al., EMBO J (1996) 15:2685-2694; Liston, P. et al., Nature (1996) 379:349-353), and promote abnormally prolonged cell survival in the developmentally-regulated model of the Drosophila eye, in vivo (Hay, B. A. et al., Cell (1995) 83:1253-1262). Deletions in a IAP neuronal inhibitor of apoptosis, NAIP, were reported in 75% of patients with spinal muscular atrophy, thus suggesting a potential role of this gene family in human diseases (Roy, N. et al., Cell (1995) 80:167-178). Therapeutic and diagnostic uses of nucleic acids that encode various inhibitors of apoptosis relating to a member of the LAP family have been described in the patent literature. See, for example, International Patent Applications No. WO 97/06255, WO 97/26331, WO 97/32601, and U.S. Pat. No. 6,245,523 to Altieri. Certain oncogenes (e.g., bcl-2) rescue cells from susceptibility to apoptosis. Specifically, members of the bcl-2 gene family can act to inhibit programmed cell death (e.g., bcl-2, bcl-xL, ced-9). The uses of such genes and gene products are contemplated for use in the invention.

In various cell culture systems, it has been shown that inhibition of ICE/CED-3 family members can effectively inhibit apoptosis. For example, the compound acetyl-DEVD-aldehyde inhibited anti-Fas induced apoptosis in a T-lymphocyte cell line (Schlegel, et al., J. Biol. Chem., 271:1841, 1996; Enari, et al., Nature, 380:723, 1996). Similarly, acetyl-YVAD-aldehyde and acetyl-YVAD-chloromethylketone blocked the death of motoneurons in vitro and in vivo (Milligan, et al., Neuron, 15:385, 1995). In addition, the ICE/CED-3 family inhibitor Boc-D-(benzyl) chloromethylketone as well as crmA prevented the cell death of mammary epithelial cells that occurs in the absence of extracellular matrix (Boudreau, et al., Science, 267:891, 1995). WO 93/05071, WO 96/03982, U.S. Pat. No. 5,585,357, Revesz et al. (Tetrahedron Left. 35, 9693-9696, 1994), U.S. Pat. No. 6,184,210, along with all other references cited herein, are incorporated by reference.

Other apoptosis inhibitors include various endonuclease inhibitors, e.g. Zn²⁺ (Cohen et al., 1984, J. Immunol., 132:38-42 and Duke et al., 1983, Proc. Natl. Acad. Sci., U.S.A., 80:6361-6365) and aurintricarboxylic acid ((Telford et al., 1991, Cell Prolif., 24: 447). Other inhibitors of apoptosis include various steroids and interleukins that are reviewed by Ellis et al., 1991, Annu. Rev. Cell. Biol., 7:663-398. The latter stage of apoptosis, i.e. the induction of fission events leading to the formation of apoptosis bodies, may be inhibited by the use of microfilament-disrupting agents such as cytochalasin B and staurosporin (Cotter et al., 1992, Cancer Res., 52:997-1005). Agents which inhibit the expression of the oncogene cMyc (Shi et al., 1992, Science, 257:212-215) or cause the over expression of proto-oncogene bcl-2 (Jacobson et al., 1993, Nature, 361:365-369) can also inhibit the induction of apoptosis. As well gene transfer of the intracellular messenger molecules, Akt/protein kinase B or Survivin have been shown to result in effective inhibition of apoptosis.

Several groups have reported that BMPs (bone morphogenetic protein) inhibit proliferation and induce apoptosis in pulmonary SMCs (Zhang S et al., 2003, A J P and Morrell N W et al., 2001, Circulation). The present inventors have discovered that BMPs actually promote cell survival and thereby protect against the regression of small arterioles and loss of pulmonary microcirculation. The use of BMPs, such as BMP2 and BMP7, are contemplated for use in the invention as apoptosis inhibitors in ECs.

The present invention contemplates that the genes may be administered by systemic gene therapy or by cell based gene therapy. As used herein, systemic gene therapy refers to the insertion of genes into cells already present in the body. As used herein, cell based gene therapy refers to the insertion of cells containing certain genes into the body.

Thus the present invention contemplates the use of these and other apoptosis inhibitory therapeutics and therapies as are known in the art.

### Administration

The apoptosis inhibitors can be administered directly to the patient, e.g. by direct infusion of the apoptosis inhibitor, into the vasculature intravenously, or by oral administration of an orally bioavailable anti-apoptotic compound. They can also be administered to the patient by processes of inhalation of an active compound or using gene therapy (e.g. a cDNA corresponding to the active protein desired or using viral vectors, for example, a replication-deficient recombinant virus coding for the apoptosis inhibitor is introduced into the patient by inhalation in aerosol form), or by intravenous injection of the DNA constituting the gene for the apoptosis inhibitor itself. Administration methods as used in known treatments of cystic fibrosis can be adopted.

Secreted inhibitors of apoptosis can be administered by cell-based gene therapy. In preparing cells containing an anti-apoptosis trans-gene for transfection and subsequent introduction into a patient's pulmonary system, it is preferred to start with somatic mammalian cells obtained from the eventual recipient of the cell-based gene transfer treatment of then present invention. A wide variety of different cell types may be used, including fibroblasts, endothelial cells, smooth muscle cells, stem cells, progenitor cells (e.g. from bone marrow or peripheral blood), adipocytes and others. Dermal fibroblasts are simply and readily obtained from the patient's exterior skin layers, and cultured in vitro by standard techniques. Endothelial cells are harvested from the eventual recipient, e.g. by removal of a saphenous vein and culture of the endothelial cells. Progenitor cells can be obtained from bone marrow biopsies or isolated from the circulating blood, using standard techniques, and cultured in vitro. The culture methods are standard culture techniques with special precautions for culturing of human cells with the intent of re-implantation.

It is preferred, in accordance with the present invention, to use dermal fibroblasts from the patient, as the cells for gene transfer. Given the fact that the logical choice of cell types for one skilled in the art to make would be a cell type naturally found in the patient's pulmonary system, such as smooth muscle cells, the use of fibroblasts is counter-intuitive. Surprisingly, it has been found that fibroblasts are eminently suitable for this work, exhibiting significant and unexpected advantages over cells such as smooth muscle cells. They turn out to be easier to grow in culture, and easier to transfect with a trans-gene, given the appropriate selection of technique. They yield a higher proportion of transfectants, and a higher degree of expression of the apoptosis inhibitors in vivo after introduction into the patient's pulmonary system. The anticipated greater risk with fibroblasts of possibly causing fibrosis in the pulmonary system, as compared with smooth muscle cells, has not materialized.

The re-introduction of the genetically engineered cells into the pulmonary circulation can be accomplished by infusion of the cells either into a peripheral vein or a central vein, from where they move with the circulation to the pulmonary system as previously described, and become lodged in the smallest arterioles of the vascular bed of the lungs. Direct injection into the pulmonary circulation can also be adopted. The infusion can be done either in a bolus form i.e. injection of all the cells during a short period of time, or it may be accomplished by a continuous infusion of small numbers of cells over a long period of time, or alternatively by administration of limited size boluses on several occasions over a period of time.

The administration of the anti-apoptosis therapeutic or a pharmaceutically acceptable salt thereof may be by way of oral, inhaled, peritoneal or parenteral administration.

An amount effective to treat the disorders hereinbefore described depends on the usual factors such as the nature and severity of the disorders being treated and the weight of the mammal. However, a unit dose will normally contain for example 0.01 to 10 mg, of the anti-apoptosis therapeutic, or a pharmaceutically acceptable salt thereof. Unit doses will normally be administered once or more than once a day, depending on the half life, for example 2, 3, or 4 times a day, more usually 1 to 3 times a day, such that the total daily dose is normally in the range of 0.0001 to 1 mg/kg; thus a suitable total daily dose for a 70 kg adult is 0.01 to 50 mg, for example 0.01 to 10 mg or more usually 0.1 to 10 mg.

The anti-apoptosis therapeutic or a pharmaceutically acceptable salt thereof is administered in the form of a unit-dose composition, such as a unit dose oral, parenteral or by inhaled composition.

Such compositions are prepared by admixture and are suitably adapted for oral, inhaled or parenteral administration, and as such may be in the form of tablets, capsules, oral liquid preparations, powders, granules, lozenges, reconstitutable powders, injectable and infusible solutions or suspensions or suppositories.

Tablets and capsules for oral administration are usually presented in a unit dose, and contain conventional excipients such as binding agents, fillers, diluents, tableting agents, lubricants, disintegrants, colourants, flavorings, and wetting agents. The tablets may be coated according to well known methods in the art.

Suitable fillers for use include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

These solid oral compositions may be prepared by conventional methods of blending, filling, tableting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminum stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or coloring agents.

Oral formulations also include conventional sustained release formulations, such as tablets or granules having an enteric coating.

Compositions of the anti-apoptosis therapeutic may be presented for administration to the respiratory tract as a snuff or an aerosol or solution for a nebulizer, or as a microfine powder for insufflation, alone or in combination With an inert carrier such as lactose. In such a case the particles of active anti-apoptosis therapeutic suitably have diameters of less than 50 microns, preferably less than 10 microns, more preferably between 2 and 5 microns.

For parenteral administration, fluid unit dose forms are prepared containing the anti-apoptosis therapeutic and a sterile vehicle. The anti-apoptosis therapeutic, depending on the vehicle and the concentration, can be either suspended or dissolved. Parenteral solutions are normally prepared by dissolving the anti-apoptosis therapeutic in a vehicle and filter sterilizing before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum.

Parenteral suspensions are prepared in substantially the same manner except that the anti-apoptosis therapeutic is suspended in the vehicle instead of being dissolved and sterilized by exposure to ethylene oxide before suspending in the sterile vehicle. Advantageously, a surfactant or wetting agent is included in the composition to facilitate uniform distribution of the anti-apoptosis therapeutic of the invention. Where appropriate, small amounts of bronchodilators for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH, may be included.

As is common practice, the compositions will usually be accompanied by written or printed directions for use in the medical treatment concerned.

A form of the method of the invention is that wherein the anti-apoptosis therapeutic or a pharmaceutically acceptable salt thereof, or a pharmaceutically acceptable solvate thereof, is administered by inhalation. It will also be appreciated from the above that it is a further aspect of the present invention to provide the treatment of disorders associated with pulmonary hypertension, by inhalation of any such vasodilator.

### Early Intervention of Pulmonary Hypertension

The discovery of a genetic defect underlying some cases of familial and sporadic PPH has provided insight into the molecular mechanisms of this disease. Specifically, mutations of the BMPR2 gene are associated familial pulmonary hypertension (Nichols W C, Koller D L, Slovis B, et al. Nat Genet. 1997;15:277-280; Lane K B, Machado R D, Pauciulo M W, et al. Nat Genet. 2000;26:81-84; Deng Z, Morse J H, Slager S L, et al. Am J Hum Genet. 2000;67:737-744). In combination with the above described invention, this knowledge can be used to facilitate the use of diagnosis and of treatments to prevent endothelial cell apoptosis and promote their survival. Thus the invention encompasses the use of such preventative measures in asymptomatic family members harboring the BMPR2 mutation.

Diagnosis of early onset of pulmonary hypertension may be carried out by, for example, assessing a lung issue biopsy for caspase immunoreactivity, using the methods taught herein. The finding by the present inventors that apoptosis is an indicative of early onset of a pulmonary disorder allows the detection of conditions involving an increase in apoptotic activity or expression. Accordingly, the present invention provides a method of detecting a condition associated with pulmonary disorders comprising assaying a pulmonary sample for (a) a nucleic acid molecule encoding an apoptosis protein or a fragment thereof or (b) dead cells or fragments thereof. In one embodiment, the condition associated with decreased pulmonary disorders is pulmonary hypertension.

### EXAMPLES

### Example 1

### Early Loss of Microvessels and Significant Increase in Endothelial Cell Apoptosis Associated with Onset of Pulmonary Hypertension

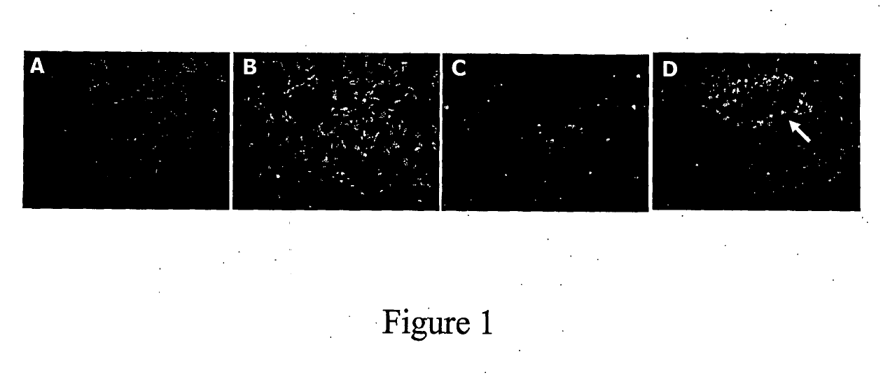

Apoptosis was studied by fluorescent TUNEL assay using confocal microscopy, as shown in FIG. 1. In the normal lung, there is only very rare or no evidence of endothelial cell (EC) apoptosis (panel A). However, only 24 hours after administration of monocrotaline (MCT), apoptosis could be clearly seen, spatially co-localized mainly to ECs of capillaries and small arterioles (panel B). This wave of programmed EC death persisted for at least 3 weeks post MCT administration (panels C and D). By the end of the experimental protocol, TUNEL positivity can be seen more in the medial sections of the larger pulmonary vessels (arrow, panel D).

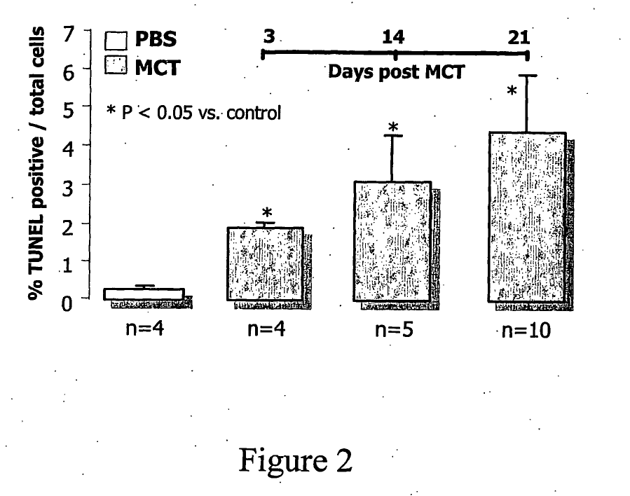

Summary data of quantitative analysis of TUNEL positive cells compared to total number of cells are shown in FIG. 2. Nuclear morphology was examined by labeling with propridium iodide.

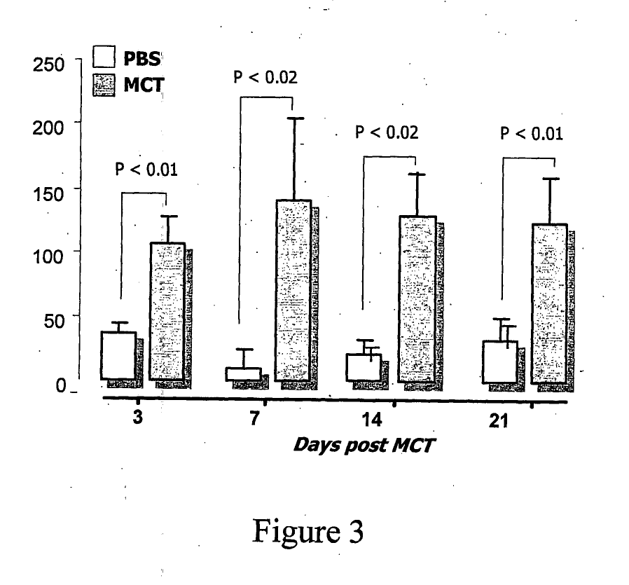

In order to get a global understanding of extend of apoptosis within the lung samples studied, a Caspase-3 activity assay utilizing whole lung lysates was used. FIG. 3 shows the Caspase-3 activity in normal (PBS treated) and MCT treated lungs. Caspase-3 activity was determined in controls, and in MCT treated rat lungs. Administration of MCT significantly increased apoptosis at all time points compared to the control group. Data represent means ±SD of n=4 animals per group. This dramatic, and previously unrecognized response to MCT suggests that apoptotic loss of microvascular endothelium may be a major mechanism of microvascular loss in pulmonary hypertension, and that this may be occurring far earlier than previously thought.

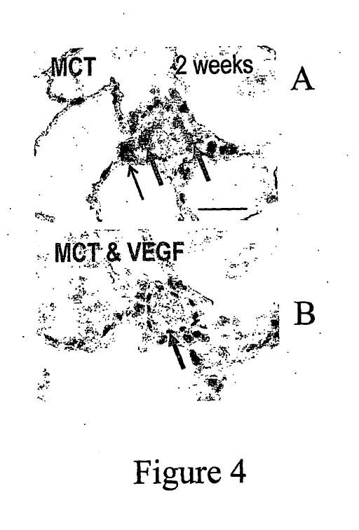

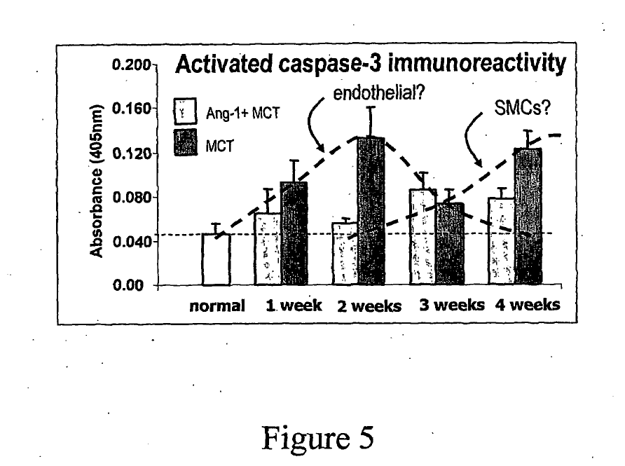

FIG. 4A shows representative examples of active caspase-3 immunostaining of lung sections 2 weeks after MCT administration. In rats receiving MCT alone, there was widespread staining of the microvascular endothelial cells localized mainly to the pre-capillary arterioles. Apoptosis of vascular cells was studied at 1, 2, 3 and 4 weeks after MCT treatment alone (n=2 to 3). As shown in FIG. 5, lungs from animals given MCT alone, staining for active caspase-3 was observed, particularly localized to endothelial cells in the smaller arterioles, and was greatest at 2 weeks, whereas smooth muscle cells only exhibited caspase-3 positivity at 4 weeks.

Given the success of their gene therapy methods, which are the subject of U.S. patent application Ser. No. 09/404,652 filed Sep. 24, 2001, the contents of which are incorporated herein by reference, the present inventors sought to determine the different mechanisms by which this treatment may work, including the stimulation of growth of new blood vessels or the inhibition of microvascular loss. One of the major limitations in pursuing this line of investigations is the lack of adequate methods for visualizing the pulmonary microcirculation. The usual light microscopy sections of the lung provide very little information about the number or 3-dimensional structure of the terminal arterioles and capillary bed, which are vital for vascular function.

The present inventors developed a method to visualize the pulmonary microcirculation by 3-dimensional confocal fluorescent microscopy. The lung is perfused with fluorescently-labeled microspheres suspended in agarose that forms a high fidelity cast of the entire arteriolar and capillary bed. Thick (200 micrometer) sections of lung are then examined by confocal microscopy, which provides stacks of ultrathin optical slices through the tissue which can then be projected or reconstructed to reveal the 3-D architecture of the pulmonary microcirculation. Therefore, this allows for the first time the direct visualization of changes in the capillary density and the number and structure of distal arterioles during the course of onset of pulmonary hypertension.

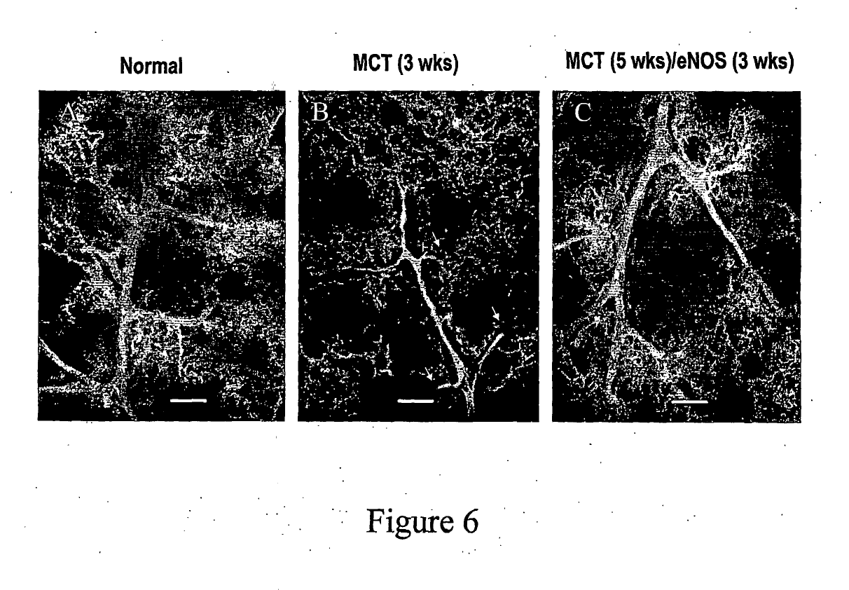

FIGS. 6A and 6B shows animal lungs 3 weeks after treatment with MCT. In the normal lung, (FIG. 6A) highly ordered architecture is apparent, with a gradual tapering of pulmonary arteriolar diameter giving rise to a profusion of evenly spaced capillaries of uniformly high density. In contrast, animals 3 weeks after treatment with monocrotaline showed a dramatic attenuation of the pulmonary arterioles which appeared thin and stretched, with marked decrease in branching (FIG. 6B). As well, there was widespread occlusion of distal pre-capillary arterioles with consequent reduction in the density of the capillary bed. These definite changes in the architecture of the microcirculation were seen even though there was only a modest degree of pulmonary hypertension (RVSP ˜35 mm Hg), at this stage.

### Example 2

### Treatment of Pulmonary Hypertension with a Survival Factor, VEGF

The full-length coding sequence of VEGF₁₆₅ was generated by reverse transcription-polymerase chain reaction (RT-PCR) using total RNA extracted from human aortic smooth muscle cells and the following sequence-specific primers: sense, 5′-TCGGGCCTCCGAAACCATGA-3′; anti-sense, 5′-CCTGGTGAGAGATCTGGTTC-3′. This generated a 649-bp fragment that was sequenced and cloned into the expression vector pcDNA 3.1 (Invitrogen) at the EcoR1 restriction site, and correct orientation was determined by use of a differential digest. The insert-deficient vector (pcDNA 3.1) was used as a control for the MCT experiments. Smooth muscle cells were transfected by use of Superfect (Qiagen Inc) with either pcDNA 3.1 or pVEGF and were then trypsinized and divided into aliquots of 500 000 cells. Six- to 8-week-old. Fisher 344 rats were injected with saline to establish normal hemodynamic and morphometric parameters (n=7). Experimental animals were injected with 80 mg/kg MCT SC (Aldrich Chemical Co) either alone (n=9) or together with 500,000 pVEGF-(n=11) or pcDNA 3.1-(n=10) transfected cells delivered via a catheter in the external jugular vein. At 28 days after injection, the animals were reanesthetized, and RV systolic pressure (RVSP) and systemic arterial pressure (SAP) were recorded in a Millar microtip catheter inserted into the RV ascending aorta. Before the catheter was placed into the aorta, 0.5 mL of arterial blood was drawing into a heparinized syringe and immediately analyzed for pH, PCO₂, PO₂, and oxygen saturation with a blood gas analyzer. The animals were then killed and the hearts excised. The RV to left ventricular (LV) plus septal weight ratios (RV/LV ratio) were determined as an indicator of hypertophic response to long-standing pulmonary hypertension L lungs were flushed via the pulmonary artery and gently insufflated with 2% paraformaldehyde via the trachea. The RV systolic pressures and RV/LV ratios were compared between the pVEGF, pcDNA 3.1, and MCT-alone groups.

To determine the effect of cell-based gene transfer of VEGF₁₆₅ on established pulmonary hypertension, 6- to 8-week-old Fisher 344 rats were injected subcutaneously with 80 mg/kg of MCT. Fourteen days after MCT injection, the animals were anesthetized, a Millar catheter was passed into the RV, and the RV pressure was recorded. Pulmonary artery smooth muscle cells transfected with either pVEGF (n=10) or pcDNA 3.1 (n=8) were then injected in aliquots of 500,000 cells into the external jugular vein, and the animals were allowed to recover. At 28 days after MCT injection (14 days after gene transfer), the animals were reanesthetized, and RVSP, SAP, and RV/LV ratios were determined as described above.

RNA extracted from rat lungs was quantified, 5 μg of total RNA from each animal was reverse-transcribed, and an aliquot of the resulting cDNA was amplified by PCR using the following sequence-specific primers: sense, 5′-CGCTACTGGCTTATCGAAATTAATACGACTCAC-3′; antisense, 5′-GGCCTTGGTGAGGTTTGATCCGCATAAT-3′, for 30 cycles with an annealing temperature of 65° C. The upstream primer was located within the T7 priming site of the pcDNA 3.1 vector, and the downstream primer was located within exon 4 of the coding region of VEGF, thus selectively amplifying a 480-bp fragment only in the presence of exogenous pVEGF mRNA. To amplify the total cellular VEGF transcript, i.e., both exogenous and endogenous, a second aliquot of the same reverse-transcription reaction was amplified with the following primers: sense (located within the 5′ UTR of the VEGF transcript), 5′-TCGGGCCTCCGAAACCATGA-3′; anti-sense (located within exon 8), 5′-CCGCCTCGGCTTGTCACATCT-3′; for 32 cycles with an annealing temperature of 62° C., generating a 589-bp fragment. Finally, a third aliquot of the same reverse-transcription reaction was amplified with the following primers for the constitutively expressed gene GAPDH: sense, 5′-CTCTAAGGCTGTGGGCAAGGTCAT-3′, antisense, 5′-GAGATCCACCACCCTGTTGCTGTA-3′. This reaction was carried out for 25 cycles with an annealing temperature of 58° C. In all cases, 10 μL of a 50-μL reaction were run on a 1.5% agarose gels.

Similar measurements were made in the experiments in which VEGF gene transfer was given 2 weeks after MCT (i.e., delayed gene transfer), except that the images were collected with a Cool-SNAP high-resolution CCD camera (Roper Scientific) and analyzed with Scion Image (Scion Corp) morphometric software. In addition, in the delayed gene transfer group, only the VEGF and MCT-alone groups were compared.

Lungs were harvested from rats treated with MCT alone or together with VEGF-transfected cells at 1, 2, 3, and 4 weeks. Formaldehyde-fixed sections were cut and mounted, and immunohistochemistry was performed with an antibody for active caspase-3 (promega).

Apoptosis of vascular cells was studied at 1, 2, 3 and 4 weeks after MCT treatment alone or together with cell-based VEGF gene transfer (n=2 to 3 for each group). In lungs from animals given MCT alone, staining for active caspase-3 was observed, particularly localized to endothelial cells in the smaller arterioles, and was greatest at 2 weeks, whereas smooth muscle cells and pericytes only infrequently exhibited caspase-3 positivity. In animals treated with VEGF together with MCT, there was noticeably less endothelial caspase-3 staining, suggesting that the improvement in pulmonary hemodynamics and arteriolar remodeling induced by VEGF may be associated with reduced endothelial cell apoptosis.

FIG. 4 shows representative examples of active caspase-3 immunostaining of lung sections harvested 2 weeks after MCT alone or together with cell-based gene transfer of VEGF. In animals treated with MCT alone (top), immunostaining was seen largely localized to endothelial cells of small arterioles (thick arrows), with occasional positivity of surrounding pericytes (thin arrow), whereas in sections from animals treated with MCT and VEGF gene transfer (bottom), endothelial staining for active caspase-3 was infrequent.

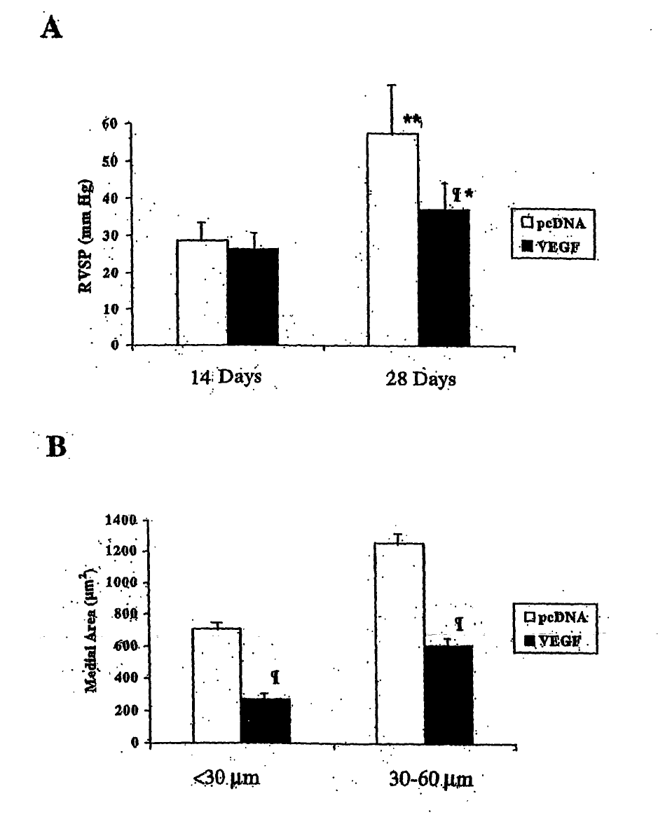

Gene therapy was delayed until two weeks after MCT treatment. FIG. 7 demonstrates RVSP at 14 and 28 days after MCT injection (A). At 14 days, pressures were determined before delivery of null-transfected (pcDNA) (n=5) or VEGF-transfected (n=7) cells. Pressures were remeasured at 28 days (2 weeks after cell-based gene therapy) in both groups. B, Medial area of pulmonary arterial vessels <30 μm and 30 to 60 μm in external diameter at 28 days for lungs from animals treated with MCT and pcDNA-transfected (n=3) or pVEGF-transfected (n=4) smooth muscle cells. *P<0.05 vs 14 days; **P<0.01 vs 14 days; ¶P<0.01 vs pDNA. Data for medial area are mean±SEM.

Data are presented as means±SD unless otherwise stated. Differences in the number of fluorescently labeled cells overtime were assessed by ANOVA, with a post hoc analysis using the Bonferroni test. All pressures, weights, arterial blood gas results, RV/LV ratios, and morphometric data were initially analyzed to determine whether the assumptions for parametric testing (normal distributions and equal variances) had been met. Because these assumptions were met for the pressure, weight, arterial blood gas, and RV/LV ratio data, differences were assessed by ANOVA, which a post hoc analysis using the t-test correlated to the number of comparisons. For the morphometric data, the assumption of normal distribution was not met for 30- to 60-μm vessel grouping. Therefore, a resampling procedure was used to test for differences between the VEGF, pcDNA 3.1, and MCT groups in both the 0- to 30- and 30- to 60-μm groups using sampling with replacement (bootstrapping) (Loughin T M, Koehler K J, Lifetime Data Anal. 1997; 3:157-177). The resampling procedure was repeated 2000 times. In all instances, a value of P<0.05 was accepted to denote statistical significance. For the morphometric data in the delayed gene transfer experiments, because only 2 groups were being compared, a Student t test was used for statistical analysis.

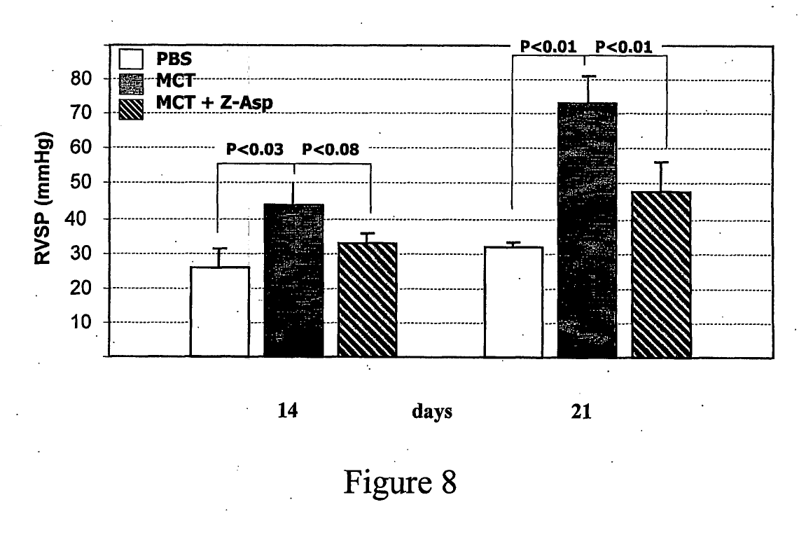

Four weeks after injection of MCT alone, RVSP was increased from 22±4 mm Hg in normal rats to 49±6 mm Hg, consistent with the development of pulmonary hypertension (P<0.001, see also FIG. 8). There was no improvement in animals receiving cells transfected with the control pcDNA 3.1 vector, with the average RVSP remaining at 48±6 mm Hg. In contrast, in animals treated with the pVEGF-transfected cells, RVSP was reduced to 32±7 mm Hg (P<0.001 versus MCT alone or null vector).

MCT-treated animals receiving pVEGF-transfected cells exhibited improved general appearance and weight gain compared with animals treated with either MCT alone or MCT in combination with the delivery of cells transfected with the null pcDNA 3.1 vector (weight increase: 71±80 versus 3±53 and 4±37 g, respectively, P<0.01). Arterial oxygen tension (PO₂) was significantly reduced in the MCT-treated rats compared with control animals (PO₂, 53±16 versus 81±9 mm Hg, respectively, P<0.01), and delivery of control vector- or VEGF-transfected cells did not result in further worsening of pulmonary gas exchange (PO₂, 65±2 and 64±6 mm Hg, respectively, P=NS versus MCT alone).

FIG. 4 shows activated caspase-3 immunoreactivity 24 hours and 2 weeks after MCT treatment. In rats receiving MCT alone, there was widespread staining of the microvascular endothelial cells localized mainly to the pre-capillary arterioles (upper panel), and this was markedly reduced in animals receiving MCT together with cell-based VEGF gene transfer (lower panel).

VEGF gene therapy using this approach was effective in inhibiting the development and progression of pulmonary hypertension and improved vascular and RV remodeling in the MCT model, even when treatment was delayed until two weeks following MCT. Particulars of VEGF transfection can also be found, for example, in the present inventors U.S. patent application Ser. No. 09/404,652 filed Sep. 24, 1999.

### Example 3

### Treatment of Pulmonary Hypertension with an Apoptosis-Inhibitor and Survival Factor, Ang1

An experiment was conducted to determine whether the now identified dramatic increase in apoptosis was causally associated with the development of pulmonary hypertension. To this end, the present inventors used Ang1, an inhibitor of apoptosis and a survival factor.

The methods set out in Example 3 were employed, except that experimental animals were injected with 80 mg/kg MCT SC. Apoptosis of vascular cells was studied at 1, 2, 3 and 4 weeks after MCT treatment alone (n=2 to 3). As shown in FIG. 5, In lungs from animals given MCT alone, staining for active caspase-3 was observed, particularly localized to endothelial cells in the smaller arterioles, and was greatest at 2 weeks (FIG. 4A), whereas smooth muscle cells only exhibited caspase-3 positivity at 4 weeks. Total lung activated caspase-3 (ELISA) was increased in MCT treated rats (n=3 per group), and this was markedly reduced by Ang1 gene transfer.

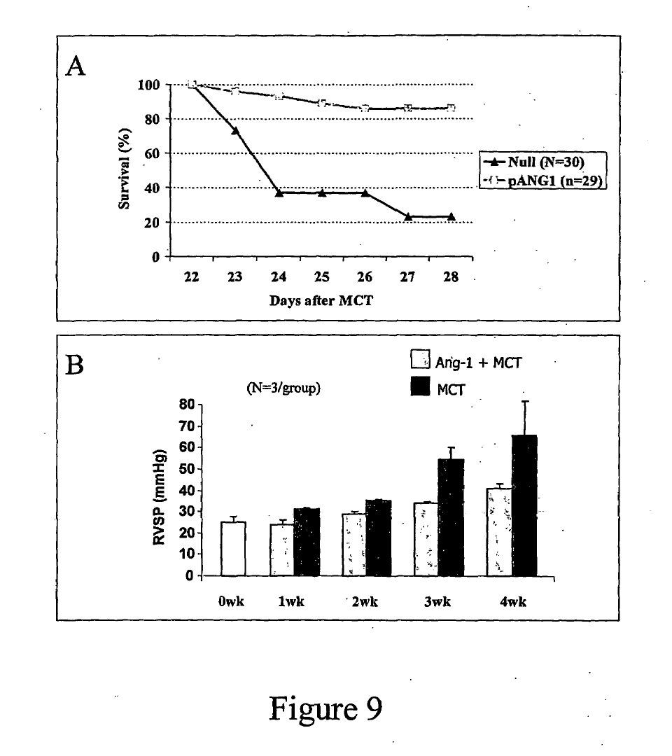

FIG. 9 shows the effect of cell-based gene transfer of Ang1 on survival (FIG. 9A) and right ventricular systolic pressure (FIG. 9B, RVSP) in the rat MCT model of pulmonary hypertension. After 21 days, null transfected rats showed significant mortality with only about 20% survival to the end of the study at 28 days. In contrast, almost 90% of Ang1 treated rats survived to the end of study. In the surviving animals, RVSP was elevated in the MCT as compared to the control rats. However, the Ang1 treated group showed significant improvement as compared with null vector controls.

### Example 4

### Treatment of Pulmonary Hypertension with Survival Factor, eNOS

An experiment was conducted determine whether the now identified dramatic increase in apoptosis was causally associated with the development of pulmonary hypertension. To this end, the present inventors used eNOS, a survival factor.

The methods set out in Example 3 were employed. Animals were treated with endothelial NO-synthase (eNOS) cell-based gene transfer at 3 weeks post MCT, and then sacrificed 2 weeks later (i.e. 5 weeks post MCT).

FIG. 6 shows 3-dimensional imaging of the pulmonary arteriolar bed using fluorescent agarose microperfusion and confocal microscopy (bar=50 mm). FIG. 6A shows the appearance of the normal rat lung. After MCT treatment (FIG. 6B) there is a marked narrowing of the distal arterioles with frequent occlusion of pre-capillary vessels (arrows). These changes were reversed as shown in FIG. 6C upon administration of eNOS.

### Example 5

### Treatment of Pulmonary Hypertension with Apoptosis-Inhibitors

Experiments were conducted to determine whether the now identified dramatic increase in apoptosis was causally associated with the development of pulmonary hypertension, and to determine more about the efficacy of treating pulmonary hypertension by blocking apoptosis and preventing pulmonary vascular damage. The time course of EC apoptosis following treatment with MCT was defined using the rat model of pulmonary arterial hypertension. In particular, the inventors determined whether the dramatic increase in apoptosis was causally associated with the development of pulmonary hypertension. To this end, the present inventors used two different class of general caspase inhibitors, namely Z-Asp (Example 6) and Z-VAD (Example 7) that are known selective inhibitors of caspase activation, proteins critically involved in mediating apoptosis.

The methods set out in Example 3 were employed. For the first set of experiments, animals were treated with Z-Asp (2 mg/rat three times per week, given intraperitoneally) or DMSO for 3 weeks beginning 3 days after administration of MCT and were sacrificed at 21 days because of obvious debilitation in the control (saline-treated) animals.

MCT treatment induced widespread apoptosis (using TUNEL assay) in the lung microcirculation, beginning at 3 days and continuing for at least 3 weeks. Microvascular apoptosis was associated with widespread capillary remodeling and microvascular loss (FIG. 1).

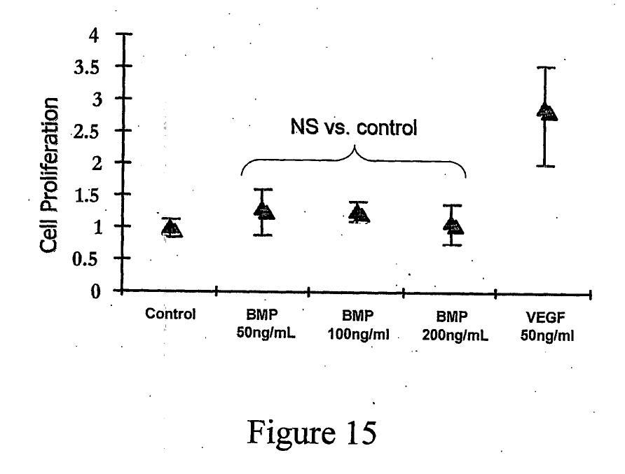

Consistent with long-standing and severe pulmonary hypertension, the RV/LV ratio was significantly elevated in animals treated with MCT alone compared to control animals (FIG. 15). In contrast, in the group receiving MCT with Z-Asp (n=10 group), the RV/LV ratio was reduced to values not significantly different from those of the control animals. Although Z-Asp treatment was begun only 3 days after administration of MCT, there was a marked and highly significant reduction in pulmonary systolic pressures in the treated group. This was also associated with reduction in the rate of apoptosis analyzed by the TUNEL assay (FIG. 5A). Based on the regression analysis there was a significant positive correlation between the prevention of pulmonary microvascular apoptosis and RVSP measurements (R²=0.296, P<0.05). Thus pulmonary hypertension was directly inhibited using Z-Asp.

### Example 6

### Treatment of Pulmonary Hypertension with Caspase Inhibitors, Z-Asp and Z-VAD

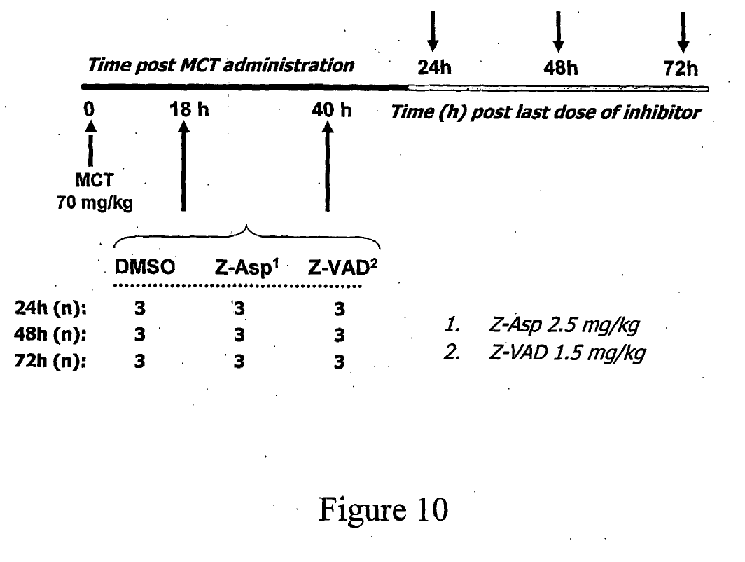

The methods set out in Example 3 were employed. In this set of experiments, the inventors tried to establish the effect of apoptosis inhibitors on the early development of the apoptotic pathways. They blocked the caspase activity at a much earlier time point (18 hours post MCT injection and another injection 24 hours after that) using two classes of caspase inhibitors, namely Z-Asp (2.5 mg/Kg) and Z-VAD (1.5 mg/Kg; see FIG. 10 for details).

Rats were sacrificed 24, 48 and 72 hours post last dose of inhibitors/DMSO injection and lungs were collected for histology and activity assay. Apoptosis were measured by both TUNEL and caspase activity assays, as previously described.

Both inhibitors were found to block the progression of apoptosis, 24, 48 and 72 hours post last dose of inhibitor based on the TUNEL assay (FIG. 911).

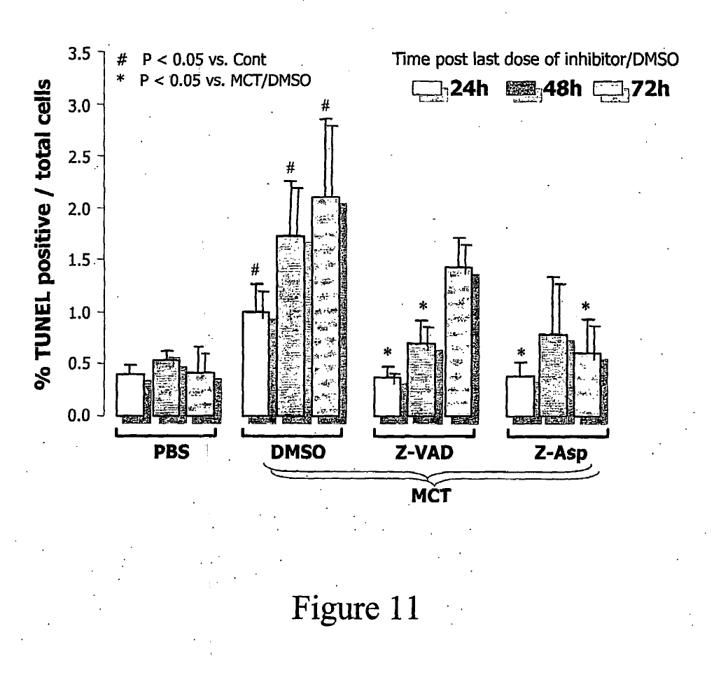

As shown in FIG. 11, inhibition of apoptosis was most effective 24 hours after the last Z-Asp or Z-VAD treatment and less potent at 48 or 72 hours. This indicates the hypothesis that apoptosis is an early event in experimental PH model; therefore, strategies to prevent the apoptotic pathways should aim at early prevention.

The inventors then investigated whether the dramatic increase in apoptosis was causally associated with the development of pulmonary hypertension. Animals were treated with Z Asp (2 mg/Kg, 3×/week) or saline for 2 weeks beginning 3 days after administration of MCT and were sacrificed at 21 days because of obvious debilitation in the control (saline treated) animals.

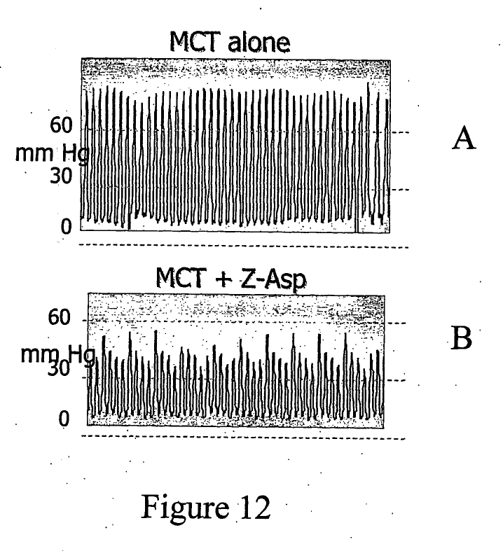

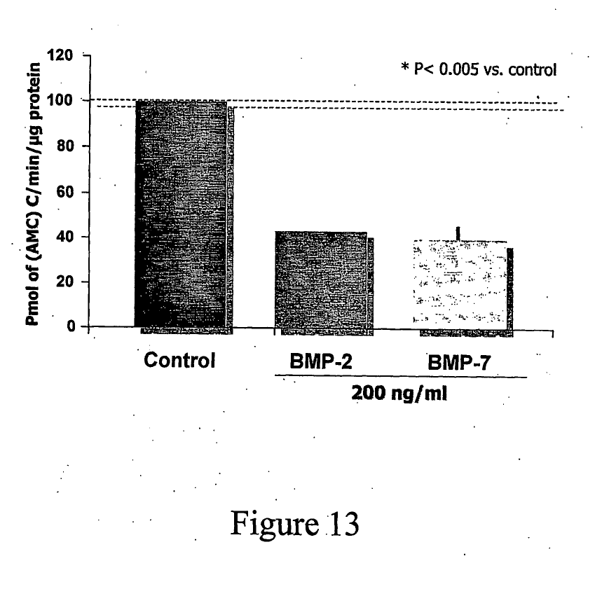

The initiation of Z-Asp treatment 3 days after administration of MCT resulted in a marked and highly significant reduction in pulmonary systolic pressures in the treated group. FIGS. 12A and 12B shows the significant increase in RSVP in MCT treated rats 21 days post MCT administration. Treatment with the caspase inhibitor Z-Asp, significantly reduced RSVP measurements compared to the non-treated MCT group. FIG. 13 summarizes the dates collected from all the experimental group (n=9). The reduction in RSVP in the Z-Asp treated group was positively associated with a reduction in the rate of apoptosis analyzed in the TUNEL assay.

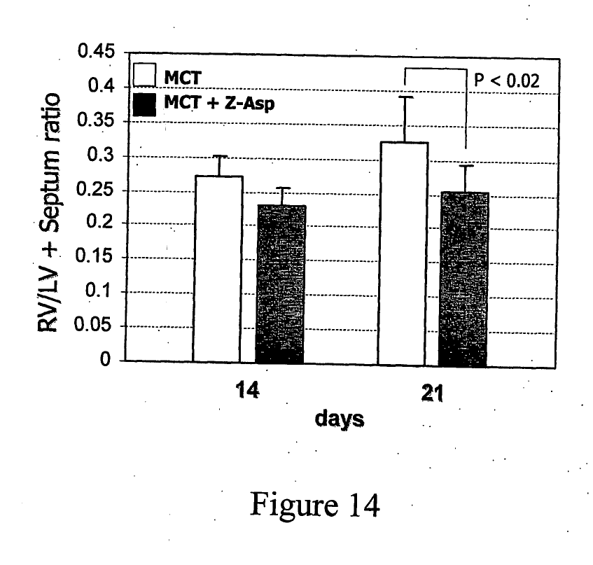

Consistent with long-standing and severe pulmonary hypertension, the RV/LV(+septum) ratio was significantly reduced in the group receiving MCT with Z-Asp (n=10 group), at both 14 and 21 day group, reaching statistical significance at the latter group (FIG. 14). This was also associated with reduction in the rate of apoptosis analyzed by the TUNEL assay (FIG. 15). Based on the regression analysis there was a significant positive correlation between the prevention of pulmonary microvascular apoptosis and RVSP measurements (R2=0.296, P<0.05).

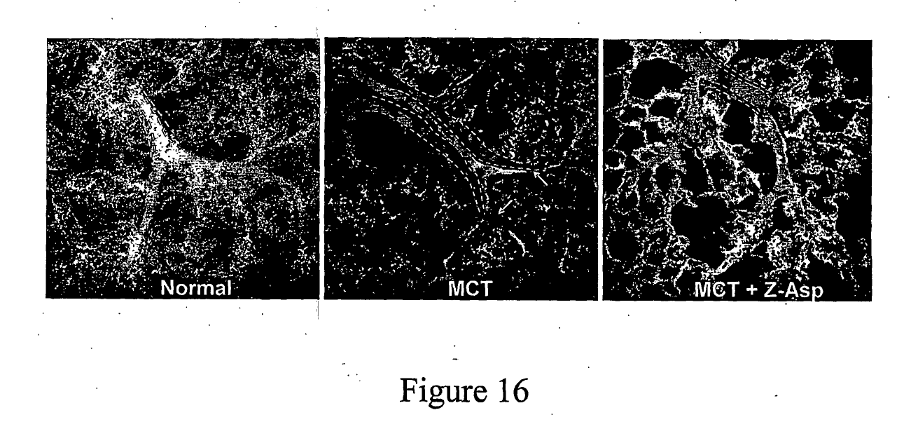

FIG. 16 shows representative photomicrographs of fluorescence microangiography of the pulmonary microcirculation, 21 days post MCT administration. Fluorescent microangiography was performed by infusion of fluorescent microspheres (0.2 μm) suspended in agarose. Lungs sections were cut on a vibratome, counterstained with propridium iodide (nuclear staining) and observed under a confocal microscope.

MCT-treated rat lungs exhibit dramatic thickening of the arterioles with marked hypoperfusion of capillaries, compared to normal lungs showing homogenous perfusion. Z-Asp treated rats showed significant improvement in overall perfusion and restoration of the capillary structures. MCT-treated rat lungs exhibited dramatic thickening of the arterioles with marked hypoperfusion of capillaries, compared to normal lungs showing homogenous perfusion. Z-Asp treated rats showed significant improvement in overall perfusion and restoration of the capillary structures.

### Example 7

### Effect of Bone Morphogenetic Protein-2 on Pulmonary Artery Endothelial Cell Proliferation and Survival

Several groups have reported that BMPs (bone morphogenetic protein) inhibit proliferation and induce apoptosis in pulmonary SMCs (Zhang S et al AJP, 2003 and Morrell N W et al, Circulation, 2001). The inventors hypothesized that BMPs promote the survival of pulmonary artery endothelial cells and thereby protect against the regression of small arterioles and loss of pulmonary microcirculation.

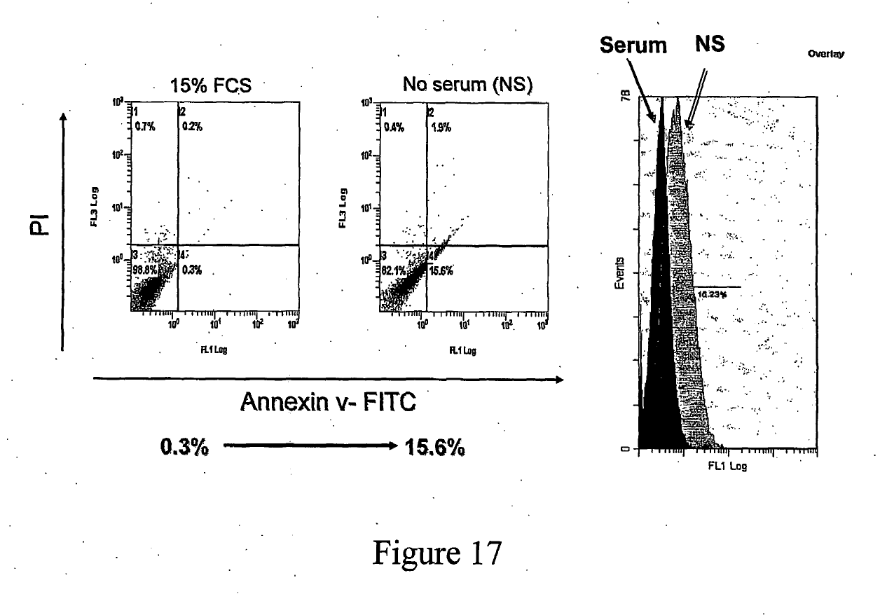

The inventors investigated the effect of BMPR2 signaling on pulmonary endothelial cell proliferation and survival. Human pulmonary artery epithelial cells (HPAEC) were grown in EBM-2 medium supplemented with 2% FBS and growth factors (Clonetics). Apoptosis was induced either by serum withdrawal (see FIG. 17) or exposure of cells to TNFα. Cells were pretreated with BMP-2 or BMP-7 (100-200 ng/ml) for 2 hours and grown in the presence or absence serum or TNF (10-20 ng/ml) for 24-48 hours. Apoptosis was evaluated by three independent methods: 1) flow cytometry using Annexin V, a Ca2+ dependent phospholipid-binding protein with affinity for phosphatidylserine (PS; translocation of PS from the inner leaflet of plasma membrane to the outer layer occurs during the early stage of apoptosis), 2) TUNEL assay and, 3) caspase activity assay.

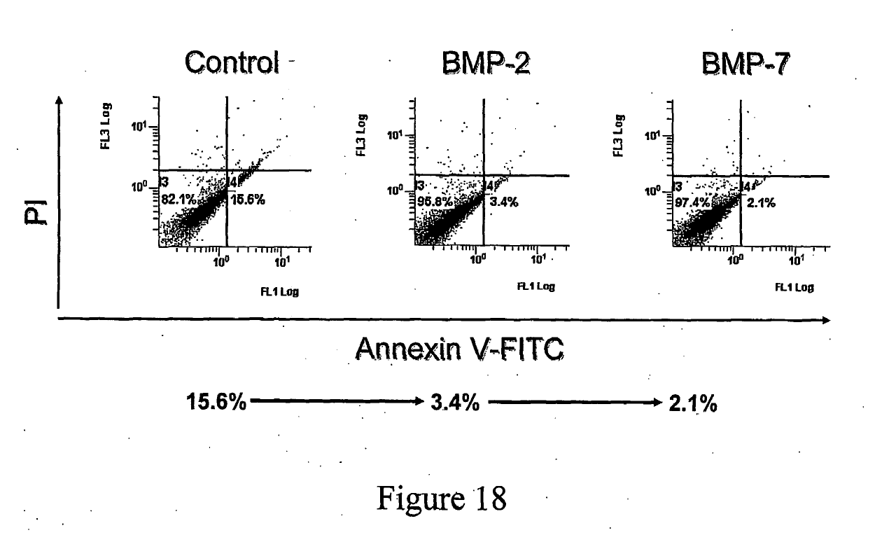

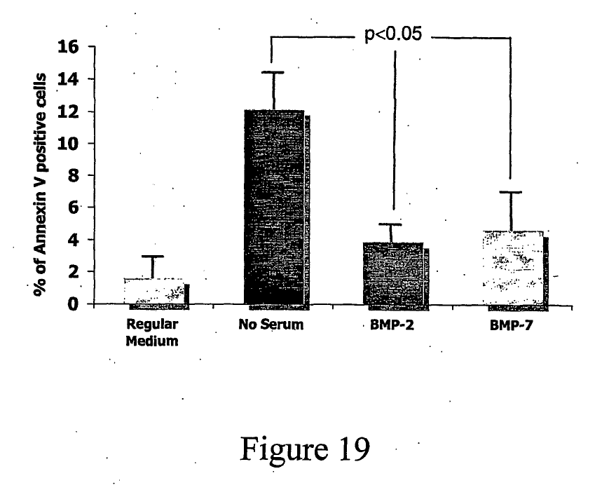

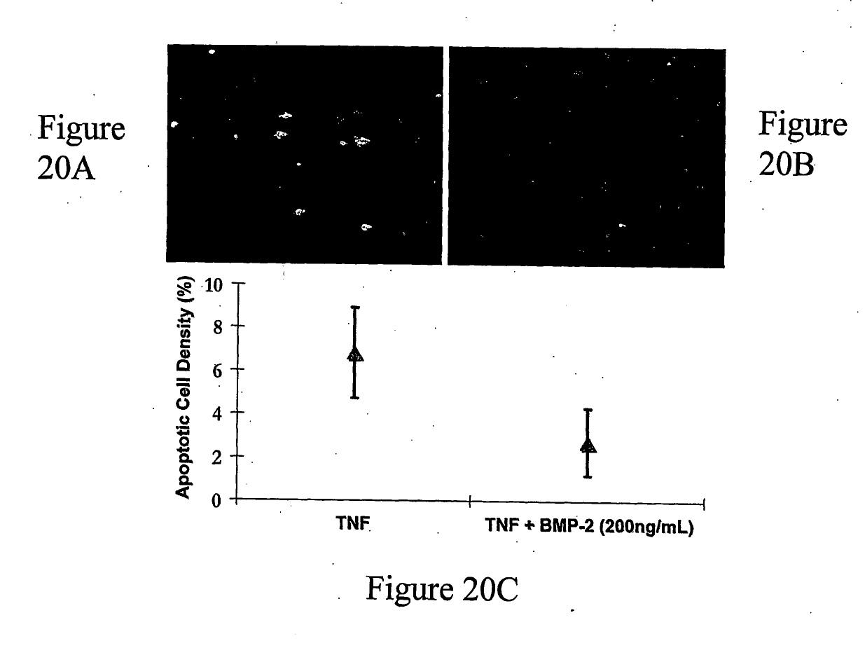

As shown in FIGS. 18, 19, and 20, pretreatment of HPAEC with BMP2 or BMP7, protected the cells from apoptosis induced by serum withdrawal as determined by flow cytometry (FIGS. 18 and 19), and TUNEL assay (FIG. 20). Pretreatment of HPAEC with BMP2 or BMP7 was also shown to protect the cells from apoptosis induced by TNF treatment as determined by the caspase activity assay (see FIG. 13).

The inventors also investigated the effect of BMPs on endothelial cell proliferation. Human endothelial cells were treated with either 50 ng/ml BMP, 100 ng/ml BMP, 200 ng/ml BMP or 50 ng/ml VEGF. Cell proliferation was determined by measuring the incorporation of ³H thymidine. Results indicate that BMPs have no effect on endothelial cell proliferation (see FIG. 15).

It is to be understood that only the preferred embodiments have been shown, and that modifications thereof would be readily apparent to one skilled in the art. Therefore, the true scope and spirit of the invention resides in the appended claims and their legal equivalents, rather than by the given examples.

1-32. (canceled)

33. A process for alleviating the symptoms of a pulmonary disorder in a mammal, comprising administering early in the onset of the disorder an effective amount of a factor selected from a group consisting of an apoptosis inhibitor and a survival factor to the pulmonary system of a mammal to alleviate a pulmonary disorder or symptoms thereof.

34. The process according to claim 33, wherein said factor is administered by systemic gene therapy.

35. The process according to claim 33, wherein said factor is administered by cell based gene therapy.

36. The process according to claim 33, wherein said factor is administered with a pharmaceutically acceptable excipient.

37. The process according to claim 33, wherein said pulmonary disorder is pulmonary hypertension.

38. The process according to claim 33, wherein said factor is delivered using viable, transfected mammalian cells, said transfected mammalian cells containing at least one expressible trans-gene coding for an apoptosis inhibitor.

39. The process according to claim 38, wherein the mammalian cells are selected from the group consisting of dermal fibroblasts, smooth muscle cells, progenitor cells, endothelial progenitor cells, epithelial progenitor cells, smooth muscle progenitor cells, stem cells, and endothelial cells.

40. The process according to claim 33, wherein said apoptosis inhibitor is selected from the group consisting of Z-Asp, Z-VAD, VEGF, Bcl-2, Bcl-xL, acetyl-DEVD-aldehyde inhibitor, acetyl-YVAD-aldehyde, acetyl-YVAD-chloromethylketone, Boc-D-(benzyl) chloromethylketone, cmA, Zn²⁺, aurintricarboxylic acid, cytochalasin B, NO, eNOS, NNOS, iNOS, NO-donor compounds, ANG1, Akt, AIP, and BMP (bone morphogenetic protein).

41. The process according to claim 40, wherein the apoptosis inhibitor is Z-Asp.

42. The process according to claim 40, wherein the apoptosis inhibitor is Z-VAD.

43. The process according to claim 40, wherein the factor is VEGF.

44. The process according to claim 40, wherein the apoptosis inhibitor is ANG1.

45. The process according to claim 35, wherein the factor is selected from the group consisting of VEGF, eNOS, iNOS, NNOS, NO-donor compounds, NO, and ANG1.

46. The process according to claim 33, wherein said mammal is human.

47. A process for preventing symptoms of a pulmonary disorder in a mammal, comprising administering a factor selected from the group consisting of an apoptosis inhibitor and a survival factor to the pulmonary system of a mammal to prevent a pulmonary disorder or symptoms thereof.

48. The process according to claim 47, wherein said factor is administered by systemic gene therapy.

49. The process according to claim 47, wherein said factor is administered by cell based gene therapy.

50. The process according to claim 47, wherein said pulmonary disorder is pulmonary hypertension.

51. The process according to claim 47, wherein said factor is delivered using viable, transfected mammalian cells, said transfected mammalian cells containing at least one expressible trans-gene coding for an apoptosis inhibitor.

52. The process according to claim 51, wherein the mammalian cells are selected from the group consisting of dermal fibroblasts, smooth muscle cells, progenitor cells, stem cells, and endothelial cells.

53. The process according to claim 47, wherein the apoptosis inhibitor is selected from the group consisting of Z-Asp, Z-VAD, VEGF, Bcl-b 2, Bcl-xL, acetyl-DEVD-aldehyde inhibitor, acetyl-YVAD-aldehyde, acetyl-YVAD-chloromethylketone, Boc-D-(benzyl) chloromethylketone, crmA, Zn²⁺, aurintricarboxylic acid, cytochalasin B, NO, eNOS, iNOS, NNOS, NO-donor compounds, AN1, Akt, AIP, and BMP (bone morphogenetic protein).

54. A pharmaceutical composition for alleviating the symptoms of a pulmonary disorder in a patient in need thereof, comprising a carrier and an apoptosis inhibitor.

55. A process for early diagnosis of a pulmonary disorder in a mammal, comprising assessing apoptosis in the pulmonary system of a mammal, wherein apoptosis is indicative of early onset of said pulmonary disorder.

56. The process according to claim 55, wherein said pulmonary disorder is pulmonary hypertension.

57. The process according to claim 55, wherein said assessing is carried out by caspase immunoreactivity assessment.

58. A kit for alleviating the symptoms of a pulmonary disorder in a mammal, comprising an effective amount of a factor selected from the group consisting of an apoptosis inhibitor and a survival factor of the pulmonary system of the mammal and instructions for the administration thereof.

59. The kit according to claim 58, wherein said instructions describe administration by systemic gene therapy.

60. The kit according to claim 58, wherein said instructions describe administration by cell based gene therapy.

61. The kit according to claim 58, further comprising a pharmaceutically acceptable excipient.

62. The kit according to claim 58, wherein said instructions describe administration using viable, transfected mammalian cells, said transfected mammalian cells containing at least one expressible trans-gene coding for an apoptosis inhibitor.

63. The kit according to claim 62, wherein said mammalian cells are selected from the group consisting of dermal fibroblasts, smooth muscle cells, progenitor cells, endothelial progenitor cells, epithelial progenitor cells, smooth muscle progenitor cells, stem cells, and endothelial cells.

64. The kit according to claim 58, wherein said apoptosis inhibitor is selected from the group consisting of Z-Asp, Z-VAD, VEGF, Bcl-2, Bcl-xL, acetyl-DEVD-aldehyde inhibitor, acetyl-YVAD-aldehyde, acetyl-YVAD-chloromethylketone, Boc-D-(benzyl) chloromethylketone, crmA, Zn²⁺, aurintricarboxylic acid, cytochalasin B, NO, eNOS, nNOS, iNOS, NO-donor compounds, ANG1, Akt, AIP, and BMP (bone morphogenetic protein).


SEQUENCE LISTING (8 sequences)

SEQ ID NO: 1 - DNA, 20 bp, Artificial
  primer - sense
tcgggcctccgaaaccatga

SEQ ID NO: 2 - DNA, 20 bp, Artificial
  primer - antisense
cctggtgagagatctggttc

SEQ ID NO: 3 - DNA, 33 bp, Artificial
  primer - sense
cgctactggcttatcgaaattaatacgactcac

SEQ ID NO: 4 - DNA, 28 bp, Artificial
  primer - antisense
ggccttggtgaggtttgatccgcataat

SEQ ID NO: 5 - DNA, 20 bp, Artificial
  primer - sense
tcgggcctccgaaaccatga

SEQ ID NO: 6 - DNA, 21 bp, Artificial
  primer - antisense
ccgcctcggcttgtcacatct

SEQ ID NO: 7 - DNA, 24 bp, Artificial
  primer - sense
ctctaaggctgtgggcaaggtcat

SEQ ID NO: 8 - DNA, 24 bp, Artificial
  primer - antisense
gagatccaccaccctgttgctgta